(19) United States
(12) Patent Application Publication
(10) Pub. No.: US 2024/0342011 A1
(43) Pub. Date: Oct. 17, 2024

(54) TAMPON INSERTION DEVICE AND METHOD

(71) Applicant: TINA HOLDINGS, LLC, Cave Creek, AZ (US)

(72) Inventors: Alison KIGHT, Palo Alto, CA (US); Stephen ERICKSON, San Francisco, CA (US)

(21) Appl. No.: 18/681,294

(22) PCT Filed: Aug. 5, 2022
(86) PCT No.: PCT/US22/39617

§ 371 Date: Feb. 5, 2024

Related U.S. Application Data
(60) Provisional application No. 63/229,650, filed on Aug. 5, 2021.

(51) Int. Cl. A61F 13/26; A61F 13/551

(52) U.S. Cl. CPC A61F 13/266; A61F 13/55175

(57) ABSTRACT

A tampon insertion device for inserting a tampon from a tampon applicator into a vaginal canal. The tampon insertion device having: a casing having a distal end, a proximal end, and an interior; an actuator located in the interior of the casing, the actuator adapted for movement within the interior of the casing; and a handle having a passageway extending through a portion thereof, wherein the passageway is sized and configured to receive the casing therethrough, with a portion of the casing positioned within the passageway such that movement of the handle with respect to the casing causes a corresponding movement of the actuator within the casing. A method of inserting a tampon is also provided.

### CROSS REFERENCE TO RELATED APPLICATIONS

This application claims priority to U.S. Provisional Patent Application No. 63/229,650, filed on Aug. 5, 2021, entitled “Tampon Insertion Device.” The entire disclosure of the foregoing provisional patent application is incorporated by reference herein.

### BACKGROUND

Tampons are the most commonly used feminine hygiene product because they are discreet and comfortable. As opposed to bulky sanitary napkins, tampons provide a sense of cleanliness, discretion, and comfort during menstruation. Current tampons and tampon applicators require independent finger strength and dexterity for proper insertion. The user must pinch the applicator between two fingers, use forces to navigate the applicator into the vagina, maintain the original pinch force while depressing the plunger to extrude the tampon into the body, use a dexterous pinch grip again to remove the applicator from the body, and then grasp and exert tension on the external string in order to remove the tampon from the body.

As depicted and described in U.S. Pub. No. 2019/0350767, published on Nov. 21, 2019 and incorporated by reference herein, a conventional tampon/applicator assembly comprises a tampon housed inside a hollow applicator barrel. A plunger extends proximally away from the barrel, with the distal end of the plunger located inside the barrel, abutting against the proximal end of the tampon. To insert the tampon, the user must pinch the grip portion of the applicator barrel, insert the portion of the barrel distal to the grip portion into her vaginal canal (while still pinching the grip portion), and then push the plunger distally into the barrel (while still pinching the grip portion) such that the tampon is expelled from the distal tip of the barrel into her vaginal canal. The grip portion must still be pinched by the user in order to remove the applicator barrel and plunger from her vaginal canal, leaving the tampon in place.

This concentric cylinder tampon/applicator design actuated by dextrous hand motion originated in 1933, see U.S. Pat. No. 1,926,900 (Catamenial device), and is still the predominant tampon insertion mechanism for tampon applicators used today. Current tampons fail to accommodate women with limited finger strength and/or dexterity.

While a variety of devices and techniques may exist for inserting tampons, it is believed that no one prior to the inventors has made or used an invention as described herein.

### BRIEF DESCRIPTION OF THE DRAWINGS

While the specification concludes with claims particularly pointing out and distinctly claiming the invention, it is believed that the invention will be better understood from the detailed description of certain embodiments thereof when read in conjunction with the accompanying drawings. Unless the context indicates otherwise, like numerals are used in the drawings to identify similar elements in the drawings. In addition, some of the figures may have been simplified by the omission of certain elements in order to more clearly show other elements. Such omissions are not necessarily indicative of the presence or absence of particular elements in any of the exemplary embodiments, except as may be explicitly stated in the corresponding detailed description.

FIG. 1 depicts an orthogonal view of one embodiment of a tampon insertion device, in the ready position primed for receiving a tampon applicator assembly therein.

FIG. 2 is a side orthogonal view of the device of FIG. 1.

FIG. 3 is the same view as FIG. 2, but with the handle advanced distally for expelling a tampon from an applicator barrel.

FIG. 4 is a view similar to FIG. 2, but with an applicator assembly mounted to the device and the handle at its proximal position, ready to expel a tampon from the applicator by distal movement of the handle relative to the casing.

FIG. 5 is a bottom orthogonal view of the device of FIG. 1.

FIG. 6 is a bottom orthogonal view of the casing of the device of FIG. 1.

FIG. 7 is an exploded schematic view of the device of FIG. 1.

FIG. 8 is a side plan view of a tampon applicator for use with the device of FIG. 1.

FIG. 9 is a bottom plan view of the device of FIG. 1, with the tampon applicator of FIG. 8 mounted to the device.

FIG. 10 is the same view as FIG. 9, but with the handle partially advanced in the distal direction.

FIG. 11 is the same view as FIG. 9, but with the handle fully advanced in the distal direction, it being understood that FIGS. 10 and 11 do not depict the expelling of a tampon from the applicator barrel).

FIG. 12A is a cross-sectional view of the device of FIG. 10, taken along the line 12A-12A thereof.

FIG. 12B is a cross-sectional view of the casing of FIG. 6, taken along the line 12B-12B thereof.

FIG. 13 is a partial bottom orthogonal view of the casing of the device of FIG. 1.

FIG. 14 is a cross-sectional side view of the device of FIG. 1, taken along the longitudinal axis X.

FIG. 15 is an orthogonal view of the handle assembly, including the attached actuator, of the device of FIG. 1.

FIG. 16 is an exploded view of one embodiment of a casing of the device of FIG. 1.

FIG. 17 is an exploded view of one embodiment of a handle of the device of FIG. 1.

FIG. 18 is a side orthogonal view of an alternative embodiment of a tampon insertion device similar to that of FIG. 1, with the addition of a handle loop affixed to the handle.

FIG. 19 is an orthogonal view of an elastic member used to form an alternative handle loop for use with the tampon insertion device of FIG. 1

FIG. 20 is an orthogonal view of the tampon insertion device of FIG. 1 with the attached elastic member of FIG. 19.

The drawings are intended to illustrate rather than limit the scope of the present invention. Embodiments of the present invention may be carried out in ways not necessarily depicted in the drawings. Thus, the drawings are intended to merely aid in the explanation of the invention. Thus, the present invention is not limited to the precise arrangements shown in the drawings.

### DETAILED DESCRIPTION

The following detailed description describes examples of embodiments of the invention solely for the purpose of enabling one of ordinary skill in the relevant art to make and use the invention. As such, the detailed description and illustration of these embodiments are purely illustrative in nature and are in no way intended to limit the scope of the invention, or its protection, in any manner. It should also be understood that the drawings are not to scale and in certain instances details have been omitted, which are not necessary for an understanding of the present invention.

Embodiments of the present disclosure provide an apparatus for inserting tampons into the vaginal canal, particularly an apparatus that facilitates tampon insertion while reducing (or eliminating) the need for independent finger strength or mobility. More particularly, the present disclosure provides an apparatus for inserting tampons by use of a handle connected to an actuator that facilitates dispensing a tampon from its applicator as the handle is advanced along the exterior of a casing. The handle and casing are configured for reciprocal movement with respect to one another such that the casing can reciprocate within a passageway in the handle. An actuator, operatively associated with the handle, is positioned within the casing such that reciprocal movement of the handle with respect to the casing causes the actuator to expel a tampon from an applicator barrel retained by the apparatus (e.g., within the casing). Unlike conventional tampon insertion methods, embodiments of the present disclosure rely on increased actuating handle accessibility and less on finger strength and dexterity. As a result, individuals who have reduced finger strength and/or dexterity are better able to accomplish tampon insertion without (or with minimal) assistance.

Some embodiments of the present disclosure provide a reusable accessory that releasably retains a tampon/applicator assembly and allows the user to insert the tampon from its applicator into the body using arm motion, while requiring little or no finger strength or dexterity. In some embodiments, the device includes a casing having a chamber adapted to receive and retain a tampon applicator assembly therein. In one particular embodiment, an elongate, hollow casing is provided. In some embodiments, the tampon applicator barrel is specifically configured to be releasably retained within the chamber of the casing. In other embodiments, the casing is configured to retain the barrel of an off-the-shelf, conventional tampon/applicator assembly. A handle is configured for axial movement with respect to the casing. For example, in some embodiments the handle is configured for sliding movement along the exterior of the casing in a direction parallel to the tampon's line of action (i.e., the axis along which the tampon is expelled from the barrel of the applicator). In one embodiment, the handle is coupled to an actuator located inside the casing. When a tampon applicator is releasably retained at least partially within the casing, the actuator (e.g., a drive plate or other drive member of the actuator) is flush with (abutting or immediately adjacent to) a proximal and of the applicator plunger of the tampon applicator. With the barrel of the tampon applicator secured with respect to the casing, the user grasps or otherwise engages the handle, navigates the distal end of the applicator barrel to the vaginal opening and inserts the barrel into the vaginal canal up to the front (i.e., distal) end of the apparatus (i.e., such that the distal end of the casing does not enter the vaginal opening). Thereafter, the user slides the handle in the direction of her body, resulting in the dispensing of the tampon into the vaginal canal.

Other embodiments of the present disclosure are adapted for use in inserting a tampon that is not supplied with its own applicator (also known as “digital tampons”). In these embodiments, the insertion device includes a barrel (e.g., a reusable barrel) and a plunger actuated by the slidable handle of the device. In some instances, the barrel is fastened to the distal end of the device, while in other instances the barrel is removably attached to the device in a manner similar to the embodiment described above (e.g., using a clamp or other retention member or feature provided on the casing).

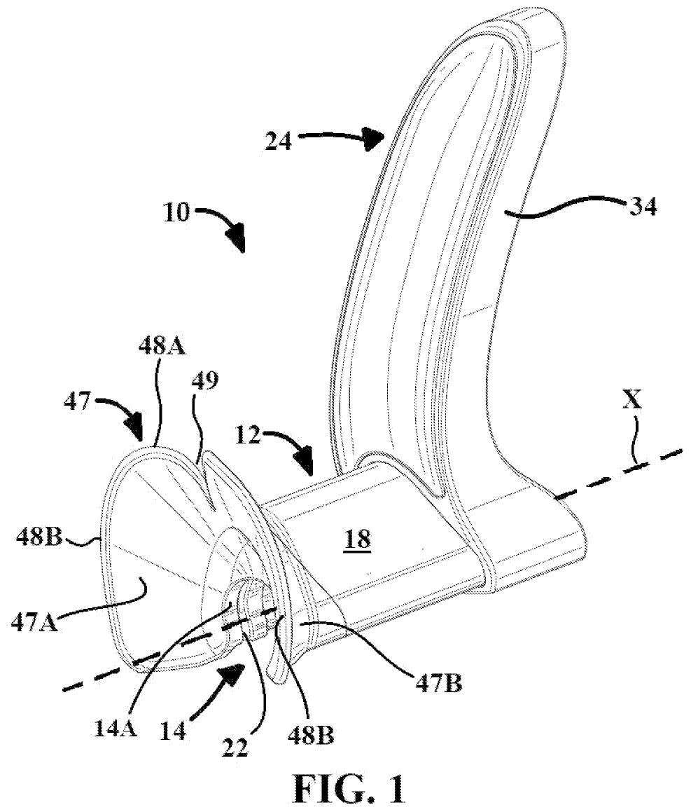

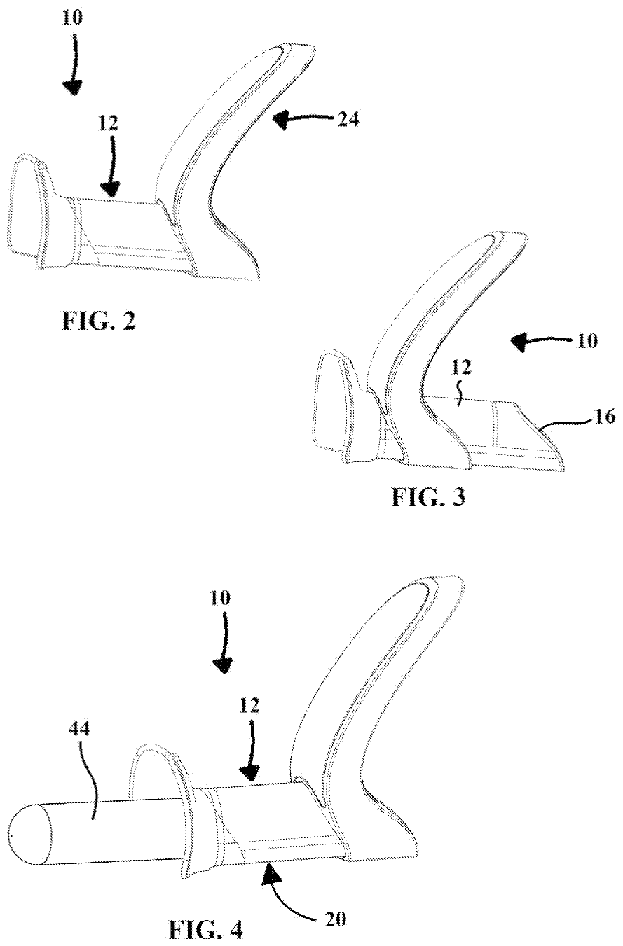

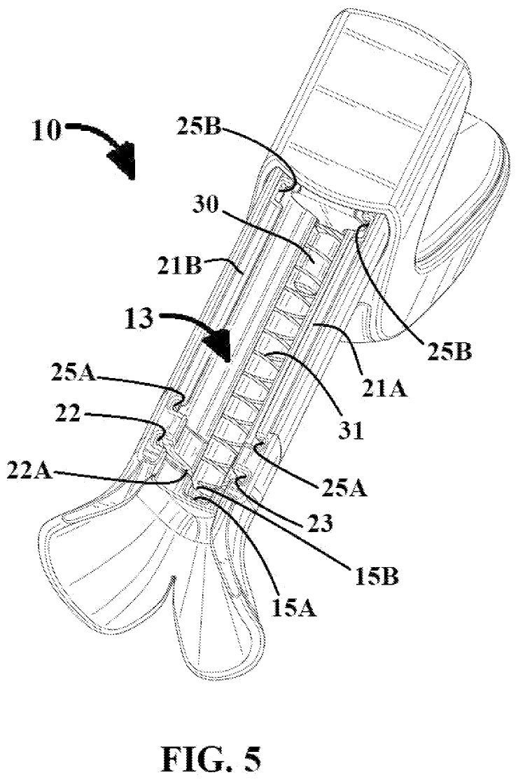

FIGS. 1-5 depict orthogonal views of one embodiment of a tampon insertion device (10). In FIGS. 1 and 2 the device is ready for insertion of a portion of a tampon/applicator assembly therein. FIG. 3 depicts the device with the handle (24) in its actuated position (i.e., for expelling a tampon). FIG. 4 is similar to FIGS. 1 and 2, but includes an applicator barrel (44) releasably mounted to the device. FIG. 5 is a bottom orthogonal view of the tampon insertion device (10).

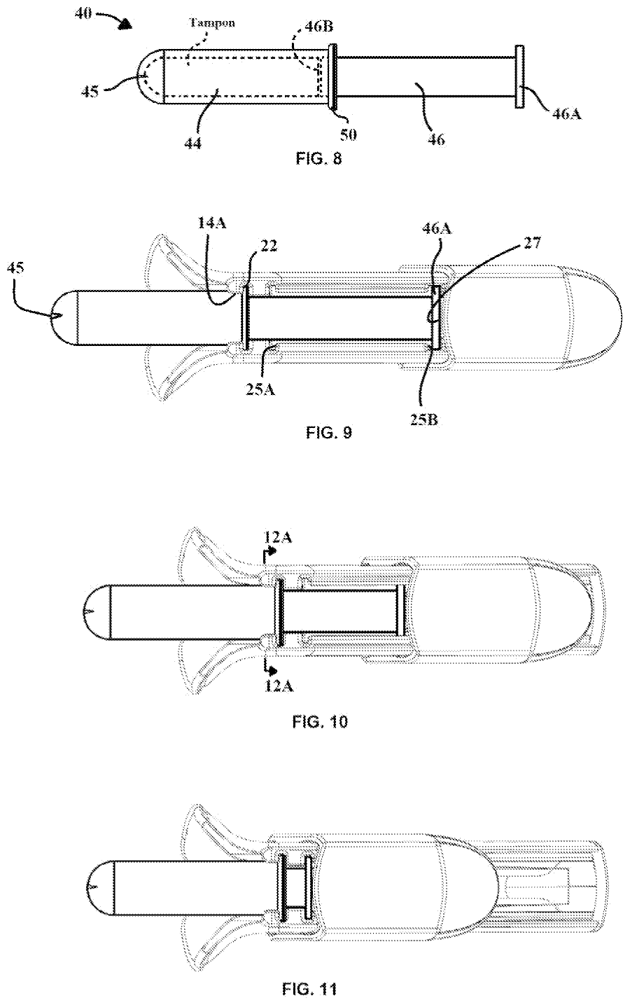

While the tampon insertion device (10) can be configured to work with any of a variety of tampon applicator designs, in the particular embodiment shown the device (10) is adapted for use with the tampon applicator (40) shown in FIG. 8. The applicator (40) includes an applicator barrel (44) in which a tampon is housed. An enlarged flange (50) having a diameter larger than that of the adjacent portion of the applicator barrel is provided at the proximal end of the barrel (44). Like conventional tampon applicators, the applicator (40) includes a plunger (46) adapted for ejecting the tampon from an opening (45) at the distal end of the barrel when the plunger is driven in the distal direction relative to the barrel. In this particular embodiment, an enlarged proximal flange (46A) having a diameter larger than that of the adjacent portion of the plunger is provided at the proximal end of the plunger (46). When the plunger (46) is driven distally into the barrel, the distal end (46B) of the plunger urges the tampon out of the barrel through opening (45). However, other arrangements of the applicator are possible, including the omission of one or both flanges (46A, 50) and/or configuring the proximal and distal ends (46A, 46B) of the plunger (46) in a variety of alternative manners.

The tampon insertion device shown includes a hollow casing (also referred to as an applicator enclosure) (12) having an open distal (or front) end (14), a proximal (or back) end wall (16) (see FIG. 3), an outer surface (18), and an open bottom (20). The casing thus defines a chamber (13) therein (see FIG. 5). A handle (24) is configured for sliding movement along the exterior of the casing (12) to expel a tampon from an applicator barrel retained within the chamber (13) of the casing. The casing (12) has a longitudinal axis (X) corresponding to the axis of actuation of the apparatus, which coincides with the axis of the tampon applicator barrel and plunger. In FIG. 1, the handle (24) is positioned at its most proximal position (at the proximal end of the casing). The handle (24) is adapted to slide along the exterior of the casing (12), parallel to the longitudinal axis (X), for expelling a tampon from an applicator barrel mounted to the device.

The casing (12) includes one or more retention features for releasably mounting a tampon applicator barrel (44) to the device, such as by releasably retaining a portion of the barrel at least partially within the chamber (13). The applicator plunger (46) is also releasably retained within the chamber (13).

The open bottom (20) of the casing is provided by spaced-apart bottom walls (21A, 21B) (see FIG. 5). In the particular embodiment shown, a groove (22) is located in the interior wall of the casing adjacent the distal end (14) extending to and through the bottom walls (21A, 21B), and is adapted to releasably retain the applicator barrel partially within the casing. Specifically, the groove (22) extends up the opposing interior sidewalls of the casing and extends around a bifurcated arcuate path orthogonal to the longitudinal axis of the casing, with the upper portion (22A) of the groove (22) intersected by a spring channel (15). The width of the groove (22) is also tapered such that the entrance (23) to the groove (22) is wider than the width of the base (22A) of the groove (see FIG. 5). This allows the flange (50) on the proximal end of the applicator barrel (44) to be captured and retained within the groove (22) by friction fit (see FIG. 9). The groove (22) also acts as a guide for ensuring that the applicator barrel is properly positioned within the casing for aligning the proximal flange (46A) of the applicator plunger (46) with an actuator located within the casing (as further explained below).

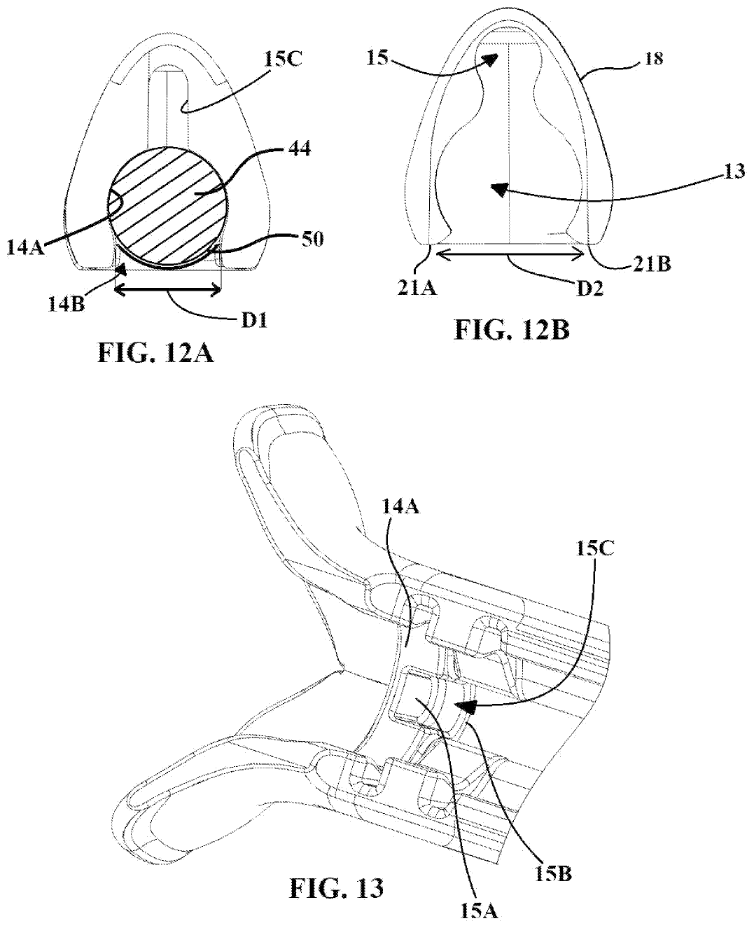

The distal end (14) of the casing, in the embodiment shown, further includes a clamp portion (also referred to as a barrel retention portion) (14A) located distal to the groove (22) (see FIGS. 1, 9 and 13). This substantially C-shaped (also referred to as U-shaped) clamp portion (14A) is partially bifurcated by reduced diameter cavity (15C) of the spring channel (15) (see FIG. 13; further explained below). The clamp portion (14A) is configured such that the tampon applicator barrel can be press-fit or clipped into the clamp portion (14A), and held in place by friction. In the embodiment shown, the clamp portion (14A) is located so that the portion of the applicator barrel (44) immediately distal to the flange (50) can be pressed or clipped into the clamp portion (14A) such that the applicator barrel is retained within the clamp portion (14A) by a friction fit, with or without a slight compression of the applicator barrel (see FIG. 12A). Thus, the interior diameter of the clamp portion (14A) is approximately the same as (or slightly smaller or larger than) the diameter of the applicator barrel (44) (see FIG. 12A). Also, the entrance (14B) to the clamp portion (14A) in the bottom of the casing has a width D1 that is slightly smaller than the diameter of the applicator barrel. This allows the applicator barrel (44) to be snapped into the clamp portion (14A), thus providing a tactile indication that the barrel has been secured within the device at the proper location (as guided by the flange (50) being received within the groove (22)). The smaller width D1 as compared to the diameter of the applicator barrel also helps to ensure that the barrel does not fall out of the casing.

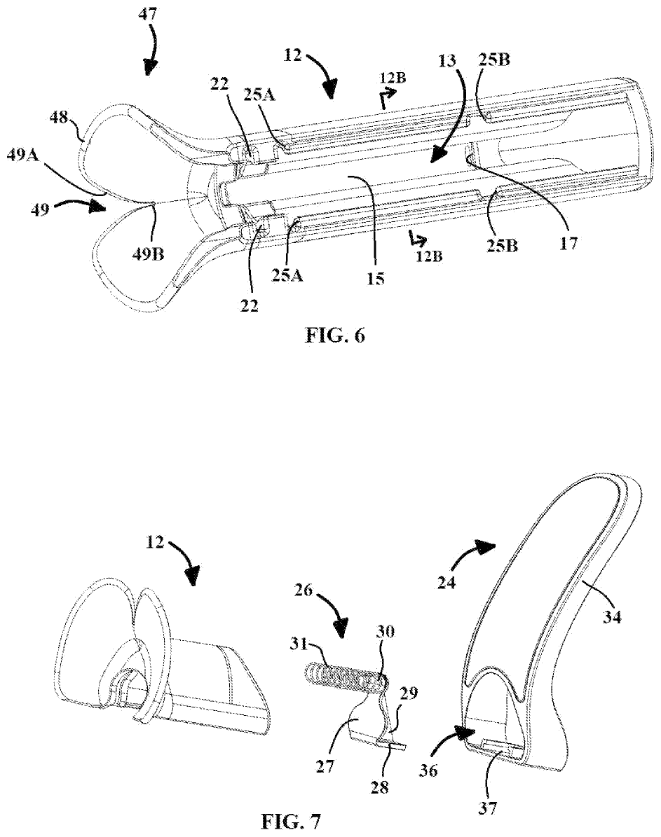

Distal slots (25A) are provided in each of the bottom walls (21A, 21B) adjacent and proximal to groove (22) (see FIGS. 5 and 6). Proximal slots (25B) are provided in each of the bottom walls (21A, 21B), proximally spaced away from the distal slots (25A). The distance between the distal and proximal slots (25A, 25B) approximately corresponds to the distance of travel of the plunger (46) within the applicator barrel (44) for ejection of a tampon. The distance (D2) between the bottom walls (21A, 21B) (i.e., the width of the opening providing access to the chamber (13)) is depicted in the cross-sectional view of FIG. 12B. Since the distance (D2) is smaller than the diameter of the proximal flange (46A) of the plunger (46), the slots (25A, 25B) allow the plunger to be inserted into and removed from the chamber (13). As also seen FIG. 12B, the interior chamber (13) of the casing is substantially C-shaped in cross-section, but bifurcated by the spring channel (15).

It will be understood that one or more of the slots (22, 25A, 25B) can be omitted, particularly when one or both of the flanges (46A, 50) are not provided on the applicator assembly (e.g., when a conventional applicator assembly is used, such as that shown in FIG. 7 of Pub. No. US 2019/0350767). In these instances, clamp portion (14A) is used to retain the applicator barrel at least partially within the casing.

As best seen in the exploded schematic view of FIG. 7, the handle (24) includes a passageway (36) sized and configured to slidingly receive the casing (12) therein. Thus, the shape of the passageway (36) generally matches that of the outer surface (18) of the casing, and is only slightly larger in order to allow for close sliding movement of the handle (24) along the outer surface of the casing. This close sliding movement stabilizes the handle (24) with respect to the casing (12) during movement.

Handle (24) further includes an elongate arm (34) extending upwardly away from the passageway (36). Arm (34) can be curved upwardly away in the proximal direction to facilitate grasping and manipulation of the arm by an end-user. Of course, arm (34) may have any of a variety of ergonomic shapes different from that shown.

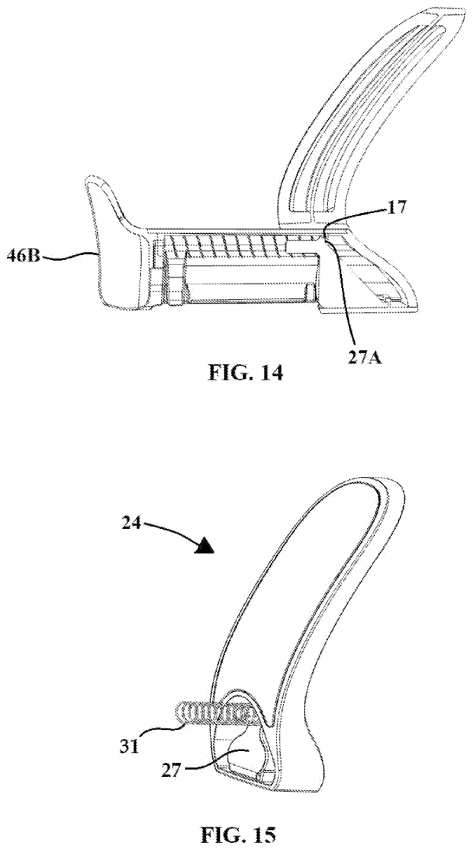

An actuator (26) is also provided for driving the plunger distally to expel a tampon from the applicator barrel. When the device (10) is assembled, the actuator (26) is located inside the chamber (13) of the casing (12), with the actuator (26) mounted for linear movement therein (i.e., movement along the line of action of a tampon/applicator assembly retained in the casing). This is best seen in the cross-sectional view of FIG. 14. The handle (24) is connected to the actuator (26) such that movement of the handle with respect to the casing (12) causes a corresponding movement of the actuator (26) within the chamber (13).

In the particular embodiment shown, the plunger actuator (26) includes a drive member such as drive plate (27), a mounting flange (28) extending proximally away from the base of the drive plate (27), and a brace (29) extending between the drive plate and the mounting flange (see FIG. 7). A spring retention pin (30) extends distally away from the upper end of the drive plate (27), parallel to the axis (X) of the device. A compression spring (31) is fit over the pin (30), extending distally parallel to the axis (X) of the device. The flange (28) of the plunger actuator (26) is received by and secured within a slot (37) located at the base of the passageway (36) of the handle (24). When the device is assembled, the upper proximal endwall (27A) of the drive plate (27) abuts against a downwardly extending abutment (17) on the interior of the casing (12) (see FIG. 14). This limits proximal movement of the handle with respect to the casing and, along with the endwall (19), prevents the handle and actuator from being detached from the casing.

The handle (24) and actuator (26) are assembled such that they reside on opposing sides of the outer wall (18) of the casing (12), with the lower portion of the handle (24) encircling the casing. With the handle (24) and actuator (26) connected (e.g., by the flange (28) of the plunger actuator (26) being secured within the slot (37) such as by an adhesive or welding), they form a subassembly that acts as a single part such that exerting a linear actuating force on the handle (24) drives the actuation of the tampon applicator plunger (46) via the plunger actuator (26).

The compression spring (31) is received within the spring channel (15) extending lengthwise in the upper wall of the chamber (13). The spring channel (15) extends from the abutment (17) to a distal end wall (15A) (see FIG. 13). The distal end portion of the spring channel (15) is stepped such that a distal spring wall (15B) is provided in the channel, proximal to the distal end wall (15A). As a result, the channel (15) includes a reduced diameter cavity (15C) at its proximal end, which receives the distal end portion of the spring retention pin (30) when the plunger actuator (26) is fully driven in the distal direction relative to the casing (FIGS. 3 and 11).

The spring (31) biases the handle and actuator in the proximal direction. As seen in FIG. 5, the distal end of the spring (31) abuts against the spring wall (15B) such that, as the plunger actuator (26) is driven in the distal direction relative to the casing using the handle arm (34), the spring (31) is compressed between the upper distal endwall of the drive plate (27) or the abutment (17) and the distal spring wall (15B) until the distal end of the retention pin (30) abuts against the distal end wall (15A) of the spring channel (15). The distal spring wall (15B) thus defines the limit of distal travel of the actuator (26) and attached handle (24). The spring acts to return the plunger actuator (26) and the handle (24) to their initial position shown in FIG. 1 when the user releases the handle (i.e., when the handle is not urged in the distal direction relative to the casing). It should be noted that the upper distal endwall of the drive plate (27) can be configured in a variety of ways so that the spring (31) is retained over the retention pin (30) secured to the drive plate.

The tampon insertion device (10) further includes an enlarged shield portion (47) that extends laterally and distally away from the open distal end (14) of the casing (12). In the embodiment shown, the enlarged shield (47) flares laterally and distally away from the open distal end (14) such that the distal face (47A) of the shield (47) is concave and the proximal face (47B) is outwardly flared (i.e., trumpeted). The shield portion (47) increases the surface area at the distal end of the tampon insertion device (10) such that the shield portion inhibits the distal (14) end of the casing (12) from entering the vaginal opening. The increased surface area provided by the shield portion (47), by not entering the vaginal opening, more efficiently facilitates an opposing normal force from the body when the handle (24) is linearly urged towards the vagina during use. These opposite forces result in a dynamic system that allows the handle (24) to be slid along the casing (12) towards the body while the flanged distal end of the casing remains relatively stationary. The distal face (47A) is also shaped to conform to a user's anatomy adjacent the vaginal opening when pressed against the curvature of the pelvis to facilitate correct orientation of the tampon applicator barrel with respect to the vaginal canal.

A string capture slot (49) is also provided in the shield (47), extending downwardly and proximally from the upper edge (48A) of the shield (47) and aligned with the longitudinal axis (X). The string capture slot can be used to assist in removing a tampon. In particular, tampon strings typically have a knot tied into the free end of the string. While the string is used to remove a tampon from the vaginal canal, this may be difficult for some women-particularly if they have limited finger strength or dexterity. The string capture slot (49) on the insertion device (10) can be used to capture the knot of the tampon string therein, thus allowing the device (10) to be used to pull the tampon out of the vaginal canal.

As best seen in FIG. 6, the string capture slot (49) is located in the shield portion (47) (although it could be located elsewhere on the device). The string capture slot (49) includes a first portion (49A) that extends downwardly and proximally from the upper edge (48A) of the shield (47), and terminates in a second portion (49B). The first portion (49A) of the capture slot tapers in width towards the second portion (49B) of the slot, while the second portion (49B) has a generally constant width that is less than that of a typical tampon string knot but slightly larger than the typical width of the untied string (e.g., about 2 to about 2.5 mm in width). To use the device (10) for tampon removal, the device is placed over the tampon string such that the string moves into the capture slot (49) and bottoms out in the second portion (49B) of the slot. The device is then slid down the removal string until the knot in the string reaches the slot (49). The user can then simply pull the device away from the vaginal opening, causing the tampon removal string to be pulled away from the user's vagina-thus removing the tampon. The capture slot (49) also serves to divide the shield portion (47) into a pair of mirror-image lobes. When combined with the outward flaring of the shield, an aesthetically pleasing appearance similar to flower petals is provided.

The positioning of the string capture slot (49) in alignment with the longitudinal axis (X) also aids in aligning the applicator barrel at the proper angle with respect to the vaginal canal. The lateral edges (48B) of the shield portion (47) also aid in ensuring that the applicator barrel is not tilted downwardly with respect to the vaginal canal during use. As seen in the cross-sectional view of FIG. 14, these lateral edges are aligned with one another and are slightly curved with respect to the line of action of the device (i.e., nearly orthogonal to the actuation plane along which the handle (24) and actuator (26) are advanced in order to expel a tampon from an applicator barrel secured within the device).

With the device in the position shown in FIGS. 1, 2, 5, and 9, the user can secure the tampon applicator (40) shown in FIG. 8 (with a tampon loaded inside the barrel) in the device (10). Specifically, the flange (50) of the barrel is urged into the slot (22) until secured therein while the proximal flange (46A) of the plunger (46) passes through the proximal slots (25B) until the plunger is positioned within the chamber (13). The casing (12) and actuator (26) are configured such that, when the handle (24) is at its proximal, retracted position (FIGS. 1, 2, 5, and 9) and the applicator barrel is properly inserted into the casing with the flange (50) secured within the groove (22), the proximal face of the flange (46A) of the plunger (46) will be positioned flush with the distal face of the drive plate (27) of the actuator (26) (see FIG. 9).

While the applicator barrel (44) can be secured to the device using two hands, the apparatus of the present disclosure is also advantageous in that this can be accomplished using only one hand, without requiring finger strength or dexterity. In particular, a tampon/applicator assembly (40) can be placed on an external surface and then, using the handle (24), the apparatus (10) is pressed over the applicator barrel (44) and plunger (46) such that the external surface on which the tampon/applicator assembly (40) rests provides the normal force necessary to snap the barrel (44) into the clamp portion (14A). To facilitate proper alignment, the proximal flange (46A) of the plunger (46) is first advanced into the entrance to proximal slots (25B) of the casing, with the distal end of the apparatus (10) angled upwardly. The apparatus is then pivoted downwardly over the proximal end of the applicator barrel such that the plunger (46) is fully received within the casing and the flange (50) and proximal end of the barrel (44) are received within the groove (22) and clamp portion (14A), respectively.

Once the applicator is loaded into the device as shown in FIGS. 4 and 9, the process of a user inserting a tampon into her vaginal canal using the tampon insertion device (10) is the same process as described in U.S. Pub. No. 2019/0350767 (and depicted in FIG. 10 therein). With the tampon/applicator assembly (40) loaded in the device (10) (i.e., in the manner shown in FIGS. 6 and 9), the user grasps or manipulates the handle (24) and navigates the device (10) and attached tampon/applicator assembly (40) towards her vagina. She then inserts the exposed portion of the applicator barrel (44) into her vaginal canal, deep enough so that the shield (47) presses against tissue adjacent (and exterior to) the vaginal opening. Next, the user simply urges the handle (24) towards her body, such as by using arm motion only-movement of the fingers is not necessary. This results in the handle (24) sliding distally along the outer surface (18) of the casing (12), since the shield (47) prevents distal movement of the casing with respect to the vaginal canal. The lateral edges (48B) and the distal face (47A) of the shield (47) provide stability and increase surface area available to translate the normal force provided by the body in the direction antiparallel to the direction of action of the handle (24) and actuator (26). These opposing forces are static on the casing (12), but dynamic as the handle (24) has freedom to slide along outer surface of the casing.

Because the handle (24) is connected to the plunger actuator (26), which is resting flush against the proximal face of the flange (46A) of the plunger (46), the distal sliding of the handle (24) along the casing urges the drive plate (27) of the actuator (26) distally against the proximal end of the applicator plunger (46) so as to push the tampon out of the applicator barrel (44) through opening (45) at the distal end of the barrel and into the vaginal canal. At this point, the tampon is positioned in the body and is no longer connected to the applicator barrel. The plunger (46) will remain in its compressed position (FIG. 11) due to friction between the plunger and the barrel interior. The device (10) can then be pulled away from the vagina. The tampon applicator barrel (44) and compressed plunger (46) (FIG. 11) are then removed from the device and the applicator (44) and plunger (46) disposed of.

As mentioned previously, the distal movement of the handle is limited by the contact of the distal end of the spring retention pin (30) with the distal spring wall (15B). This prevents the actuator (26) from pushing the applicator (40) out of the casing (12). In addition, when the handle (24) and actuator (26) are fully advanced distally (i.e., the distal end of the pin (30) abutting distal spring wall (15B)), the proximal flange (46A) of the plunger (46) will be aligned with the distal slots (25A) in the bottom walls (21A, 21B) as seen in FIG. 11. This arrangement allows the applicator to be removed from the casing as the distal slots (25A) allow the proximal flange (46A) to escape from the chamber (13).

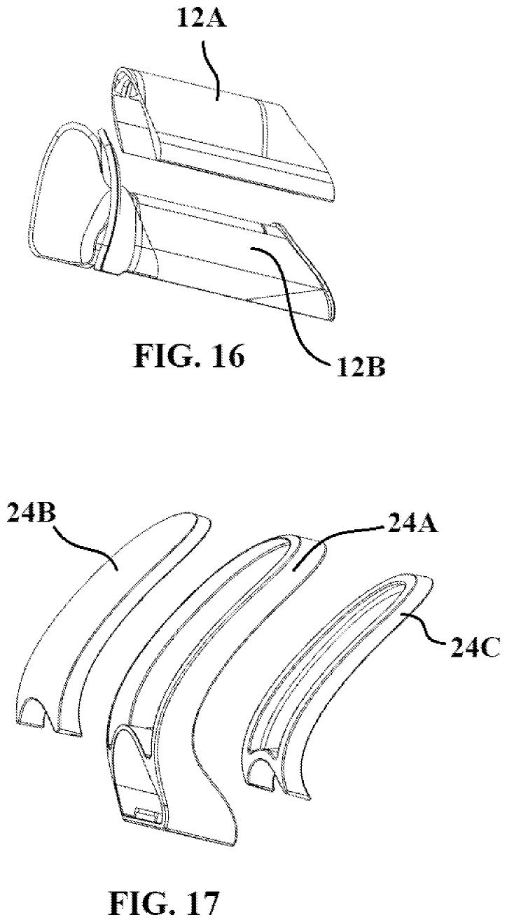

The device (10) can be manufactured in a variety of ways, particularly molding from plastic or other polymeric materials. By way of example, the casing (12) and handle assembly (24) can each be manufactured from two or more pieces using an overmolding process. As seen in FIG. 16, the casing (12) can be formed from a first portion (12A) molded from a rigid plastic (e.g., polypropylene), and a second portion (12B) overmolded over the first portion (12A) from a more pliable material such as a thermoplastic or thermoset elastomer. This provides a softer, more pliable shield portion (47) and greater friction between the walls of the chamber (13) and the exterior surfaces of the applicator, thereby helping to retain the applicator within the chamber. Similarly, as seen in FIG. 17, the handle assembly (24) can be formed from a first portion (24A) molded from a rigid plastic (e.g., polypropylene), and second and third portions (24B, 24C) overmolded over the first portion (24A) from a more pliable material such as a thermoplastic or thermoset elastomer. This provides softer, more pliable, and easier to grip surfaces on the handle (24).

While the handle (24) of FIGS. 1-7 is depicted as having an upwardly curved arm (34) extending away from the passageway (36), the handle can be configured in a variety of alternative ways intended to improve the ergonomics of device manipulation and/or actuation. For example, the handle may include a loop-shaped portion to facilitate manipulation of the handle, as described in U.S. Pub. No. 2019/0350767.

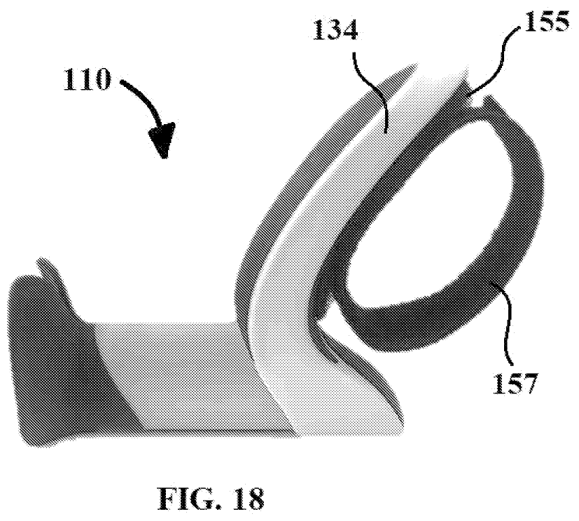

FIG. 18 depicts an alternative embodiment of a tampon insertion device (110) wherein the arm (134) of the handle has been modified from that shown in FIGS. 1-9 so as to include a loop portion (157) on the proximal face of the arm. The user can insert her hand into the loop (157), thereby facilitating manipulation of the handle, particularly for users with limited finger strength and/or dexterity. The loop allows the user to manipulate the device without grasping the handle with her fingers. In this instance, a strip (155) of hook-and-loop fastener material is secured to the proximal face of the arm, and a corresponding strip (157) of hook-and-loop fastener material is releasably and adjustably attached to the strip (155), thereby forming a loop for receiving the user's hand. This arrangement also allows the size (inner diameter) of the loop to be adjusted to provide a more snug fit with the user's hand.

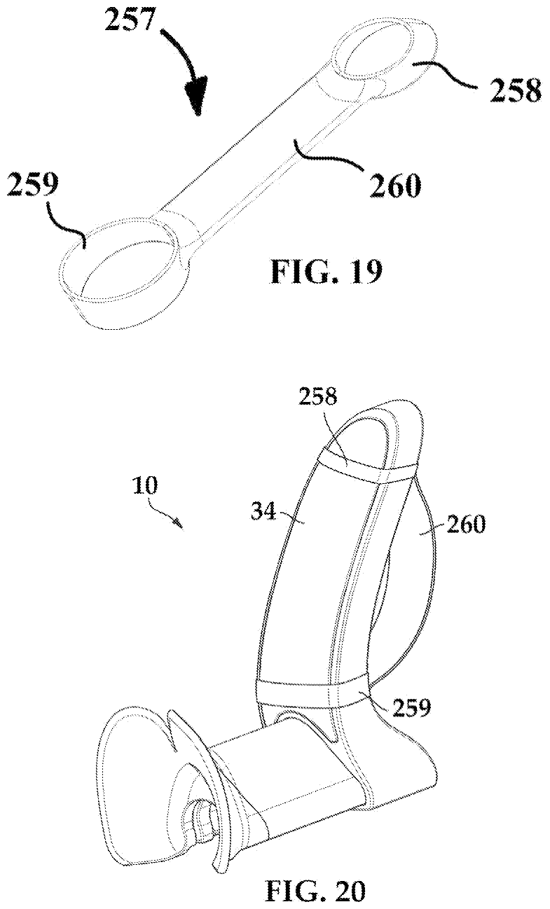

As yet another alternative, a handle loop can be provided by an elastic member (257) shown in FIGS. 19 and 20. Elastic member (257) includes a first ring (258) located at one end of an elongate strap (260), and a second end ring (259) located at the other end of the strap (260). The rings (258, 259) are sized and shaped to be removably securable on the arm (34) of the device (10), as shown in FIG. 20. Thus, the smaller first ring (258) is secured about an upper portion of the arm (34), with the larger second ring (259) secured about a lower portion of the arm (34) such that a loop is formed by the strap (260) and the proximal face of the arm (34), between the rings (258, 259). The user's hand can be inserted into this loop for manipulating the device, including advancement of the handle along the casing. Since the member (257) is elastic, the member (257) and the arm (34) can be configured to provide a loop having a size for snugly receiving the user's hand, thereby providing a more stable engagement of the arm by the user's hand to facilitate distal advancement of the arm (34) with respect to the casing.

The present invention has been described in terms of several particular embodiments and applications, both in summarized and detailed form. However, it is not intended that development of a tampon insertion aid is limited to the scope of these embodiments. It will be understood that many substitutions, changes and variations in the described embodiments, applications and details of the methods and components illustrated herein and of their operation can be made by those skilled in the art without departing from the spirit of this invention.

1. A tampon insertion device for inserting a tampon from a tampon applicator into a vaginal canal, the tampon applicator having a barrel for containing the tampon and a plunger for expelling the tampon from a distal end of the barrel, the device comprising:
(a) a casing having a distal end, a proximal end, and an interior;
(b) an actuator located in the interior of the casing, the actuator adapted for movement within the interior of the casing; and
(c) a handle having a passageway extending through a portion thereof, the handle connected to the actuator, wherein the passageway is sized and configured to receive the casing therethrough, with a portion of the casing positioned within the passageway such that movement of the handle with respect to the casing causes a corresponding movement of the actuator within the casing;
wherein the casing is adapted for releasably mounting the tampon applicator barrel to the device such that movement of the handle in the direction of the distal end of the casing causes the tampon to be expelled from the distal end of the tampon applicator barrel while retaining the applicator barrel mounted to the device.

2. The tampon insertion device of claim 1, wherein the casing is adapted for releasably mounting the tampon applicator barrel to the device such that the distal end of the barrel is spaced away from the distal end of, and external to, the casing.

3. The tampon insertion device of claim 2, wherein, the interior of the casing is adapted to receive and releasably retain a portion of the applicator barrel therein, with the applicator plunger positioned within the interior of the casing.

4. The tampon insertion device of claim 1, wherein the casing is hollow and elongate, has an open distal end, and has a longitudinal axis, and further wherein the casing is positioned within the passageway of the handle such that the handle is linearly movable along the exterior of the casing parallel to the longitudinal axis of the casing with the casing passing through the passageway of the handle.

5. The tampon insertion device of claim 4, wherein said actuator and said handle are linearly movable along an actuation plane that includes the longitudinal axis of the casing in order to expel a tampon from the applicator barrel.

6. The tampon insertion device of claim 4, wherein the casing further includes an open bottom through which the tampon applicator can be inserted into the interior of the casing.

7. The tampon insertion device of claim 6, wherein the actuator is coupled to the handle through the open bottom of the casing.

8. The tampon insertion device of claim 7, wherein the actuator comprises a drive member positioned within the casing.

9. The tampon insertion device of claim 7, wherein the drive member is connected to and extends upwardly away from the passageway of the handle into the casing.

10. (canceled)

11. The tampon insertion device of claim 1, wherein the actuator is spring-biased in the proximal direction.

12. The tampon insertion device of claim 11, further comprising a compression spring positioned within the casing and spring-biasing the actuator and handle in the proximal direction.

13-14. (canceled)

15. The tampon insertion device of claim 1, wherein the casing further comprises a clamp portion adapted to retain a portion of the tampon applicator barrel in the casing such that the distal end of the barrel is spaced away from the distal end of, and external to, the casing with a proximal end of the plunger adjacent the actuator.

16. (canceled)

17. The tampon insertion device of claim 1, wherein the casing further comprises at least one groove located in an interior wall of the casing and extending along an arcuate path, wherein the at least one groove is adapted for receiving a flange on the applicator.

18. The tampon insertion device of claim 17, wherein casing comprises a plurality of grooves located in an interior wall of the casing and extending along an arcuate path, wherein each of the grooves is adapted for receiving one or more flanges on the applicator.

19. (canceled)

20. The tampon insertion device of claim 1, further comprising an enlarged shield portion extending away from the distal end of the casing.

21. (canceled)

22. The tampon insertion device of claim 20, wherein the shield portion flares laterally and distally away from the distal end of the casing such that a distal face of the shield portion is concave a proximal face of the shield portion is outwardly flared.

23. The tampon insertion device of claim 20, wherein the shield portion includes a string capture slot.

24. (canceled)

25. The tampon insertion device of claim 1, wherein the handle further comprises an arm extending upwardly away from the passageway.

26. The tampon insertion device of claim 25, wherein the handle further comprises a handle loop for facilitating manipulation of the device, the handle loop adapted to the receive the user's hand therethrough.

27. The tampon insertion device of claim 26, further comprising an elastic member attached to the arm such that the handle loop is formed by the elastic member and a proximal face of the arm.

28-29. (canceled)

30. A method of inserting a tampon into a user's vaginal canal, comprising the steps of:
(a) providing a tampon insertion device of claim 1, with a tampon located within a tampon applicator barrel with the barrel mounted to the device;
(b) inserting the distal portion of the applicator barrel into the vaginal canal; and
(c) sliding the handle of the insertion device distally with respect to the casing and towards the vagina such that the plunger expels the tampon from the opening in the distal end of the barrel and into the vaginal canal.
